(19) United States
(12) Patent Application Publication
(10) Pub. No.: US 2026/0153492 A1
(43) Pub. Date: Jun. 4, 2026

(54) ETHYLENE SENSOR

(71) Applicant: Massachusetts Institute of Technology, Cambridge, MA (US)

(72) Inventors: Timothy Swager, Newton, MA (US); Darryl Fong, Boston, MA (US); Shaoxiong Luo, Cambridge, MA (US); Rafaela Da Silveira Andre, Cambridge, MA (US)

(73) Assignee: Massachusetts Institute of Technology, Cambridge, MA (US)

(21) Appl. No.: 18/972,595

(22) Filed: Dec. 6, 2024

Related U.S. Application Data
(63) Continuation of application No. 17/127,292, filed on Dec. 18, 2020, now abandoned.
(60) Provisional application No. 62/978,266, filed on Feb. 18, 2020.

(51) Int. Cl. G01N 33/00; B01J 27/122; B01J 31/02; B01J 31/30; G01N 27/12

(52) U.S. Cl. CPC G01N 33/0098; B01J 27/122; B01J 31/0239; B01J 31/0252; B01J 31/30; G01N 27/127; B01J 2231/005; B01J 2531/002

(57) ABSTRACT

Wacker oxidation can be used as a signal transduction mechanism for the selective and sensitive detection of ethylene in air via chemiresistive sensing. Using this system, the senescence of lisianthus flowers and carnations can be monitored.

### PRIORITY CLAIM

This application claims priority to U.S. Provisional Patent Application No. 62/978,266, filed Feb. 18, 2020, which is incorporated by reference in its entirety.

### FEDERALLY SPONSORED RESEARCH OR DEVELOPMENT

This invention was made with Government support under Grant No. DMR1809740 awarded by the National Science Foundation (NSF), and under Grant No. W912HZ-17-2-0027 awarded by the U.S. Army Engineer Research and Development Center. The Government has certain rights in the invention.

### FIELD OF THE INVENTION

The invention relates to systems and methods for detecting ethylene.

### BACKGROUND

Ethylene regulates developmental processes in flowers like ripening, secondary metabolite synthesis, seed germination/flowering, and senescence. See, for example, Janssen, S. et al. Ethylene Detection in Fruit Supply Chains. Philos. Trans. R. Soc. A Math. Phys. Eng. Sci. 2014, 372, which is incorporated by reference in its entirety. It is recognized in biological systems by a family of five transmembrane proteins (ETR1, ETR2, ERS1, ERS2, and EIN4), each of which contribute to different ethylene response pathways. See, for example, Li, H.; Guo, H. Molecular Basis of the Ethylene Signaling and Response Pathway in Arabidopsis. J. Plant Growth Regul. 2007, 26, 106-117, which is incorporated by reference in its entirety. Ethylene also plays a central role in stress-induced feedback loops. For example, basal ethylene production can be modulated in response to salinity, metals, hypoxia, air pollutants (O₃, SO₂, etc.), and pathogens. See, for example, Morgan, P. W.; Drew, M. C. Ethylene and Plant Responses to Stress. Physiol. Plant. 1997, 100, 620-630, which is incorporated by reference in its entirety. Given the central role ethylene plays in plant health, it is perhaps unsurprising that ethylene detection is of considerable interest to the agriculture industry. Indeed, it is estimated that upwards to 50% of a farm's production value may be lost as a result of various issues along the supply chain. See, for example, Salunkhe, D. K. et al. Senescence of Flowers and Ornamentals—Basic Principles and Considerations. In Postharvest Biotechnology of Flowers and Ornamental Plants; 1990; pp 13-27, which is incorporated by reference in its entirety. Ethylene monitoring could reduce losses if changes in plant health are detected at an early stage and preventative actions are taken.

### SUMMARY

In one aspect, a sensor can include a conductive region in electrical communication with at least two electrodes. The conductive region can include a conductive material and a palladium based catalyst material in contact with the conductive material.

In certain circumstances, the conductive material can include a p-type conductor or an n-type conductor.

In certain circumstances, the conductive material can include a carbon nanotube, a carbon nanotube including a coordinating group, graphite, or graphene.

In certain circumstances, the conductive material can include a metal oxide the conductive material can include a silica/zinc oxide nanoparticle and/or nanofiber.

In certain circumstances, the conductive material can include an inorganic semiconductor.

In certain circumstances, the carbon nanotube can include a coordinating group. The coordinating group can be a nitrogen heteroaryl group, for example, a pyridyl group. In one example, the conductive material can include pyridyl group functionalized carbon nanotubes For example, the coordinating group can contain pyridyl groups, for example pyridyl groups functionalized carbon nanotubes. The pyridyl group contains a nitrogen group that has an sp² electronic configuration and one skilled in the art will understand that there are a number of other groups that can a display equivalent a pyridine group. As a result, the term pyridyl is indicative of a nitrogen based group capable or interacting with a metal similarly to a pyridine. There can also be multiple points of interaction between pyridyl groups. For example, a pyridyl can be a bipyridine or terpyridine group.

In certain circumstances, the palladium based catalyst can include Pd(II). For example, the palladium based catalyst can include a Pd(II) salt and a nitrite salt, a Pd(II) salt with a Cu(II) salt and a nitrite salt, or a Pd—Cu heterobimetallic complex and a nitrite salt.

In certain circumstances, the palladium based catalyst can include a nitrite salt, for example, an alkyl ammonium nitrite salt.

In certain circumstances, the sensor can include an ionic liquid solvent.

In certain circumstances, the sensor can include an oil or flexible polymer.

In certain circumstances, the sensor can include a liquid material. For example, the liquid material can include an aromatic alcohol.

In certain circumstances, the sensor can include a polymer that has a glass transition temperature that is higher than the conditions in which the sensor is expected to operate.

In certain circumstances, the electrode can include gold or chromium.

In certain circumstances, the sensor can include a flow chamber for routing a gas from a sample to the conductive region.

In another aspect, a method of sensing an analyte can include exposing a sensor to a sample, the sensor including a conductive region in electrical communication with at least two electrodes, the conductive region including a conductive material and a palladium based catalyst material in contact with the conductive material, and measuring an electrical property at the electrodes.

In another aspect, a method of preparing a sensor can include forming a complex including a conductive region in electrical communication with at least two electrodes, the conductive region including a conductive material and a palladium based catalyst material in contact with the conductive material; and placing the conductive material in electrical communication with at least two electrodes.

In the methods described herein, the sensor can have one or more of the features described above.

In certain circumstances, the sample can include a gas.

In certain circumstances, the electrical property can include resistance or conductance.

In certain circumstances, the analyte can include one or more of ethylene, 1-methylcyclopropene, butadiene, isoprene, carbon monoxide, acetylene, an alkene or an alkyne.

In certain circumstances, the analyte can have a concentration of less than 100 ppm, less than 50 ppm, less than 1 ppm, less than 10 ppm or less than 1 ppm. For example, the analyte can have a concentration of 500 ppb or less.

In certain circumstances, the analyte can be an emission from a vegetable, fruit, flower, or plant.

Other aspects, embodiments, and features will be apparent from the following description, the drawings, and the claims.

### BRIEF DESCRIPTION OF DRAWINGS

FIGS. 1A-1C depict oxidation reactions that can be used in the sensors described herein. FIG. 1A depicts anti-Markovnikov Wacker oxidation with nitrite co-catalyst reported by Grubbs and co-workers. See, for example, Wickens, Z. K.; Morandi, B.; Grubbs, R. H. Aldehyde-Selective Wacker-Type Oxidation of Unbiased Alkenes Enabled by a Nitrite Co-Catalyst. Angew. Chemie—Int. Ed. 2013, 52, 11257-11260, which is incorporated by reference in its entirety. FIG. 1B depicts translation of catalytic aerobic Wacker oxidation for the sensitive and selective chemiresistive detection of ethylene gas. FIG. 1C depicts a schematic of sensing device containing gold electrodes on a glass substrate with a SWCNT network and liquid selector mixture.

FIGS. 2A-2B show an ethylene sensing mechanism. FIG. 2A depicts control experiments omitting [PdCl₂(PhCN)₂], ⁿBu₄N[NO₂], benzyl alcohol (BA), or oxygen. (6,5) SWCNTs only and benzyl alcohol with (6,5) SWCNTs were also tested. FIG. 2B depicts sensing curves using p-type (6,5) SWCNTs (green trace) and n-type SiO₂/ZnO nanofibers (purple trace) as the semiconducting material. Devices were exposed to 50 ppm of ethylene in air for 1 min (N≥6).

FIGS. 3A-3B show investigation of the difference in sensing response when using reaction mixtures with and without CuCl₂. FIG. 3A depicts sensing responses for optimized conditions (Pd=[PdCl₂(PhCN)₂], Cu=CuCl₂, and nitrite=ⁿBu₄N[NO₂]) and (6,5) SWCNTs only. FIG. 3B depicts Raman spectra collected using 532 nm excitation wavelength with spectra intensity normalized to the G-band.

FIGS. 4A-4B show sensing response using (6,5) SWCNTs functionalized covalently with 4-pyridyl or phenyl groups (0.7-1.4 functional groups per 100 C atoms) or noncovalently with P4VP. Devices were exposed to 50 ppm of ethylene in air for 1 min (N≥6).

FIGS. 5A-5C show sensitivity and stability of the ethylene sensor. In each condition, the reaction mixture contains 120 mM [PdCl₂(PhCN)₂] and 90 mM ⁿBu₄N[NO₂] in benzyl alcohol. (FIGS. 5A and 5B) Devices were exposed to ethylene in air for 1 min (N≥6). FIG. 5C depicts sensor response before and after storage for 16 days at 4° C.

FIG. 6 shows selectivity in the presence of various interferents. The reaction mixture contains 120 mM Pd ([PdCl₂(PhCN)₂]) and 90 mM nitrite (ⁿBu₄N[NO₂]) in benzyl alcohol. Device response after exposure to VOCs in air for 1 min using 4-pyridyl functionalized SWCNTs (N≥6). The y-axis for VOCs is expanded by 20×.

FIGS. 7A-7C show flower senescence monitored using our sensory system. The reaction mixture contains 120 mM [PdCl₂(PhCN)₂] and 90 mM ⁿBu₄N[NO₂] in benzyl alcohol. Device response after exposure to flower volatiles in air for 1 min using 4-pyridyl functionalized SWCNTs (N≥6). FIG. 7A depicts a response to purple lisianthus flowers and red carnations over several days and FIG. 7B depicts a response to red carnations treated with nothing (orange trace), water only (blue trace), or water treated with the nutrient package (red trace). FIG. 7C depicts photographs of lisianthus flowers and carnations over time.

FIGS. 8A-8B show solvent vs sensor response (−ΔG/G₀). FIG. 8A depicts devices containing 120 mM [PdCl₂(PhCN)₂], 120 mM CuCl₂, and 60 mM ⁿBu₄N[NO₂] in solvent (benzyl alcohol, 1-decanol, ethylene glycol, 2-phenyl-2-propanol, PEG₄₀₀, or tetrahydromyrcenol) were exposed to 50 ppm of ethylene in air for 1 min (N≥3). FIG. 8B depicts a plot of −ΔG/G₀ vs total oxidation yield (refer to Table 1).

FIGS. 9A-9C show optimization of the [PdCl₂(PhCN)₂]:CuCl₂:ⁿBu₄N[NO₂] stoichiometry using benzyl alcohol as solvent. When optimizing a given reagent, the other two ingredients were held at constant concentration. Sensing response upon exposure to 50 ppm of ethylene in air for 1 min with varying concentrations of (FIG. 9A) [PdCl₂(PhCN)₂], (FIG. 9B) ⁿBu₄N[NO₂], and (FIG. 9C) CuCl₂ (N≥3).

FIG. 10 shows ¹H NMR spectra overlay in CDCl₃ of 1-heptene (blue), and the crude reaction mixture after 16 h under ambient conditions using 3.9 mol % [PdCl₂(PhCN)₂] and 2 mol % ⁿBu₄N[NO₂] with benzyl alcohol (red) or toluene (green) as solvent.

FIGS. 11A-11B show optimization of ⁿBu₄N[NO₂] concentration using benzyl alcohol as the solvent. [PdCl₂(PhCN)₂] was held constant at 120 mM. Samples were exposed to 50 ppm of ethylene in air for 1 min (N≥8). FIG. 11A depicts sensing curves and FIG. 11B depicts response vs ⁿBu₄N[NO₂] concentration.

FIGS. 12A-12B show optimization of the (FIG. 12A) transducer material (using [PdCl₂(PhCN)₂]) and (FIG. 12B) Pd source using benzyl alcohol as the solvent. 120 mM Pd(II) and 90 mM ⁿBu₄N[NO₂] were used for each experiment. Devices were exposed to 50 ppm of ethylene in air for 1 min (N≥6). dppe=ethylenebis(diphenylphosphine); dppf=1,1′-bis(diphenylphosphino)ferrocene; (NHC)₂Pd₂Cl₄=dichloro(di-μ-chloro)bis[1,3-bis(2,6-di-i-propylphenyl)imidazol-2-ylidene]dipalladium(II); XPhos Pd G2=chloro(2-dicyclohexylphosphino-2′,4′,6′-triisopropyl-1,1′-biphenyl)[2-(2′-amino-1,1′-biphenyl)]palladium(II).

FIGS. 13A-13B show characterization of SiO₂/ZnO nanofibers. FIG. 13A depicts a FEG-SEM that reveals smooth nanofibers with a mean diameter of 160±60 nm and no observable bead defects. FIG. 13B show a HR-TEM that reveals that the nanofibers are composed of crystalline nanoparticles with interplanar distances of 2.45 Å, corresponding to the d-spacing of the (101) plane in hexagonal ZnO.

FIG. 14 shows response upon multiple 1 min exposures to 50 ppm ethylene in air using SiO₂/ZnO nanofibers as an n-type semiconductor (N≥6). The reaction mixture contains 120 mM [PdCl₂(PhCN)₂] and 90 mM ⁿBu₄N[NO₂] in benzyl alcohol.

FIGS. 15A-15B show Raman spectra collected at 633 nm of (FIG. 15A) 4-pyridyl and (FIG. 15B) phenyl functionalized (6,5) SWCNTs. Spectra were normalized to the G-band at ˜1590 cm⁻¹.

FIGS. 16A-16C show survey XPS spectra of (6,5) SWCNTs covalently functionalized 4-pyridyl groups.

FIG. 17 shows TGA data showing mass loss upon heating under a nitrogen atmosphere of phenyl-functionalized (6,5) SWCNTs.

FIG. 18 shows investigation of the n-doping effect upon exposure of 4-pyridyl functionalized (6,5) SWCNTs to Pd(0) generated in situ (Pd=[PdCl₂(PhCN)₂] and nitrite=ⁿBu₄N[NO₂]). Raman spectra were collected using 532 nm excitation wavelength with spectra intensity normalized to the G-band.

FIGS. 19A-19B show sensitivity using pristine (6,5) SWCNTs. In each condition, the reaction mixture contains 120 mM [PdCl₂(PhCN)₂] and 90 mM ⁿBu₄N[NO₂] in benzyl alcohol. Devices were exposed to ethylene in air for 1 min (N≥6).

FIG. 20 shows sensitivity vs relative humidity (R.H.) normalized to the response at 2% R.H. for comparison of 4-pyridyl functionalized and pristine (6,5) SWCNTs. Devices were exposed to 50 ppm ethylene in air for 1 min (N≥6).

FIGS. 21A-21B show a comprehensive stability study of (a) 4-pyridyl (covalently) functionalized and (b) pristine (6,5) SWCNTs. In each condition, the reaction mixture contains 120 mM [PdCl₂(PhCN)₂] and 90 mM ⁿBu₄N[NO₂] in benzyl alcohol. Devices were exposed to 50 ppm ethylene in air for 1 min (N≥6).

FIGS. 22A-22B show selectivity using pristine (6,5) SWCNTs. In each condition, the reaction mixture contains 120 mM [PdCl₂(PhCN)₂] and 90 mM ⁿBu₄N[NO₂] in benzyl alcohol. Devices were exposed to analyte in air for 1 min (N≥6). Selectivity (FIG. 22A) in the presence of various interferents and (FIG. 22B) in the presence of various alkenes. In FIG. 22A, the y-axis for VOCs is expanded by 8×.

FIG. 23 shows a schematic for the sensing experiment setup to analyze flower volatiles. Mass flow controllers (MFCs) are used to control the overall flow rate to 200 mL/min.

FIGS. 24A-24B show representative photographs of (a) lisianthus flowers and (b) carnations as received.

FIGS. 25A-25B show representative photographs of the populations for (FIG. 25A) lisianthus flowers and (FIG. 25B) carnations after one week of growth in nutrient water.

### DETAILED DESCRIPTION

Ethylene is a dynamic plant hormone and its temporal monitoring can be used to glean insight into plant health and status. However, the real-time distributed detection of ethylene at trace levels under ambient conditions remains a challenge. A single-walled carbon nanotube-based chemiresistor catalyst combination that can detect ppb levels of ethylene in air. Cycling between Pd(II) and Pd(0) via Wacker oxidation with a nitrite co-catalyst imparts response discrimination driven by the chemoselectivity of the chemical transformation. Sensitivity is controlled by a combination of the chemical reaction efficiency and the n-doping strength of the Pd(0) species generated in situ. The covalent functionalization of the carbon nanotube sidewall with pyridyl ligands drastically improves device sensitivity via enhanced n-doping. The utility of this ethylene sensor is demonstrated in the monitoring of senescence in red carnations and purple lisianthus flowers.

Ethylene detection is typically accomplished using photoacoustic spectroscopy or gas chromatography. See, for example, De Gouw, J. A. et al. Airborne Measurements of Ethene from Industrial Sources Using Laser Photo-Acoustic Spectroscopy. Environ. Sci. Technol. 2009, 43, 2437-2442; and Pham-Tuan, H. et al. Automated Capillary Gas Chromatographic System to Monitor Ethylene Emitted from Biological Materials. J. Chromatogr. A 2000, 868, 249-259, each of which is incorporated by reference in its entirety. Although these methods are sensitive, these analytical tools are impractical for real-time and/or in-field measurements. Other ethylene detection methods include turn-on fluorescence with luminescent polymers or olefin metathesis catalyst/fluorescent dye hybrid molecules, chemiresistors, graphene-based field-effect transistors, electrochemical oxidation, and amperometry. See, for example, Esser, B.; Swager, T. M. Detection of Ethylene Gas by Fluorescence Turn-on of a Conjugated Polymer. Angew. Chemie—Int. Ed. 2010, 49, 8872-8875; Green, O. et al., AgBF4-Impregnated Poly(Vinyl Phenyl Ketone): An Ethylene Sensing Film. J. Am. Chem. Soc. 2004, 126, 5952-5953; Toussaint, S. N. W. et al. Olefin Metathesis-Based Fluorescent Probes for the Selective Detection of Ethylene in Live Cells. J. Am. Chem. Soc. 2018, 140, 13151-13155; Sun, M. et al. Rapid and Visual Detection and Quantitation of Ethylene Released from Ripening Fruits: The New Use of Grubbs Catalyst. J. Agric. Food Chem. 2019, 67, 507-513; Vong, K., et al. An Artificial Metalloenzyme Biosensor Can Detect Ethylene Gas in Fruits and Arabidopsis Leaves. Nat. Commun. 2019, 10, 5746; Esser, B. et al. Selective Detection of Ethylene Gas Using Carbon Nanotube-Based Devices: Utility in Determination of Fruit Ripeness. Angew. Chemie—Int. Ed. 2012, 51, 5752-5756; Fu, W. et al. Ultrasensitive Ethene Detector Based on a Graphene-Copper(I) Hybrid Material. Nano Lett. 2017, 17, 7980-7988; Zevenbergen, M. A. et al. Electrochemical Sensing of Ethylene Employing a Thin Ionic-Liquid Layer. Anal. Chem. 2011, 83, 6300-6307; and Jordan, L. R. et al. Portable Trap-Sensor System for Monitoring Low Levels of Ethylene. Analyst 1997, 122, 811-814, each of which is incorporated by reference in its entirety. In each of these reports, there is some combination of cumbersome sensor preparation, low sensitivity, humidity intolerance, and/or sensitivity to oxygen. Given these drawbacks, there is an unmet need for the real-time robust monitoring of trace ethylene under diverse ambient conditions.

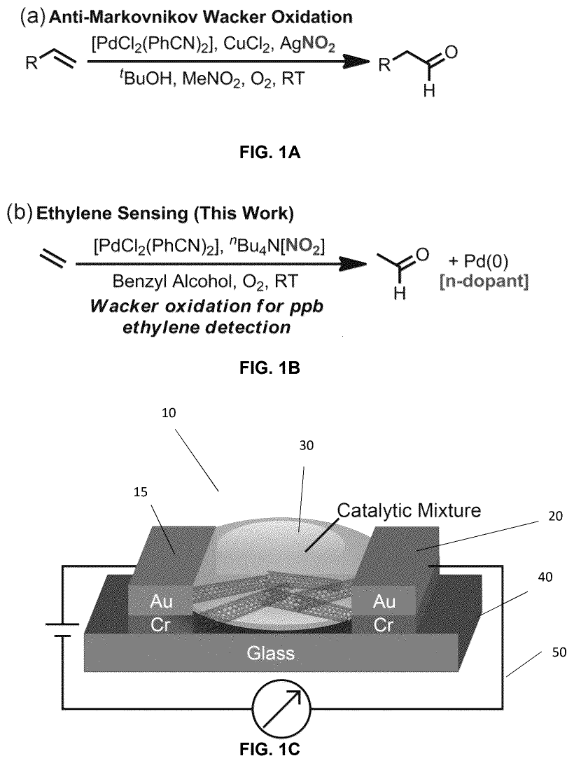

In this work, a highly selective ethylene chemiresistive sensor that can detect ppb levels of ethylene under ambient conditions. The sensor is prepared from commercially available materials and leverages the sensitivity of single-walled carbon nanotubes (SWCNTs) conductance to carrier densities. See, for example, Schroeder, V. et al. Carbon Nanotube Chemical Sensors. Chem. Rev. 2019, 119, 599-663, which is incorporated by reference in its entirety. Specifically catalytic aerobic ethylene oxidation is employed, wherein the palladium catalyst toggles between electron-rich Pd(0) and electron-poor Pd(II) to reversibly modulate the degree of p-doping in the SWCNTs. See, for example, Schroeder, V.; Swager, T. M. Translating Catalysis to Chemiresistive Sensing. J. Am. Chem. Soc. 2018, 140, 10721-10725, which is incorporated by reference in its entirety. Our method was inspired by the anti-Markovnikov Wacker oxidation initially reported by Grubbs and co-workers, which uses a nitrite co-catalyst to achieve selective olefin oxidation under mild conditions (FIGS. 1A-1B). See, for example, Wickens, Z. K. et al. Aldehyde-Selective Wacker-Type Oxidation of Unbiased Alkenes Enabled by a Nitrite Co-Catalyst. Angew. Chemie—Int. Ed. 2013, 52, 11257-11260, which is incorporated by reference in its entirety. The utility of our ethylene sensor by monitoring plant senescence has been demonstrated, which is a process that is mediated by low ppb concentrations of ethylene.

In one aspect, a sensor can include a conductive region in electrical communication with at least two electrodes. The conductive region can include a conductive material and a palladium based catalyst material in contact with the conductive material. The catalyst converts the analyte to a product. For example, the catalyst can be a Wacker oxidation catalyst, capable of oxidizing an olefin to a ketone or aldehyde.

The palladium based catalyst material can include a non-coordinating anion, for example, Cl⁻, ClO₄⁻, BF₄⁻, RSO₃⁻ where R is CF₃, CH₃, an aryl, an alkyl, or an oxygen bound alkyl or aryl group, PF₆⁻, or BAr₄⁻, where Ar is an aromatic group (for example, the alkyl or aryl groups can be C1-C8 alkyl or C6-C14 aryl or heteroaryl groups). For example, the palladium based catalyst material can include palladium(II) nitrile compounds or palladium(II) chloride compounds.

The resistivity or conductivity of the sensor can change when the sensor is exposed to an analyte. A conductive material conducts electricity. The conductive material can include a metal, an organic material, a dielectric material, a semiconductor material, a polymeric material, a biological material, a nanowire, a semiconducting nanoparticle, a nanofiber, a carbon fiber, a carbon particle, carbon nanotubes, graphite, graphene, carbon paste, metal particles, or conducting ink, or combination thereof. The conductive material can include an organic electronic material, a conductive polymer, a doped conjugated polymer, or a conductive inorganic material.

A conductive polymer can include a poly(fluorene), a polyphenylene, a polypyrene, a polyazulene, a polynaphthalene, a poly(pyrrole) (PPY), a polycarbazole, a polyindole, a polyazepine, a polyaniline (PANI), a poly(thiophene) (PT), a poly(3,4-ethylenedioxythiophene) (PEDOT), a poly(p-phenylene sulfide) (PPS), a poly(acetylene) (PAC), a poly(p-phenylene vinylene) (PPV), or copolymers thereof. A metal oxide can include ZnO₂, SnO₂, TiO₄, WO₃, MoO₃, NiO, SnO, or combinations thereof. The inorganic semiconductor can include SiO₂/ZnO₂, MoS₂, MoSe₂, ZnS₂, Si, Ge, InP, or combinations thereof. The inorganic semiconductor can be a nanofiber.

In certain circumstances, the conductive material can include a p-type conductor or an n-type conductor. For example, the conductive material can include a carbon nanotube, graphite, graphene, the alkene-interacting metal complex, a conductive polymer, a metal oxide, or an inorganic semiconductor. In certain circumstances, the conductive material can include a metal oxide the conductive material can include a silica/zinc oxide nanoparticle and/or nanofiber. In certain circumstances, the conductive material can include an inorganic semiconductor.

In certain circumstances, the conductive material can include a carbon nanotube, a carbon nanotube including a coordinating group, graphite, or graphene. A nanostructure can be an articles having at least one cross-sectional dimension of less than about 1 μm, less than about 500 nm, less than about 250 nm, less than about 100 nm, less than about 75 nm, less than about 50 nm, less than about 25 nm, less than about 10 nm, or, in some cases, less than about 1 nm. Examples of nanostructures include nanotubes (e.g., carbon nanotubes), nanowires (e.g., carbon nanowires), graphene, and quantum dots, among others. In some embodiments, the nanostructures include a fused network of atomic rings. The conductive material can be a single-walled carbon nanotubes (“SWNT”), double-walled carbon nanotubes, semi-conductor quantum dots, semi-conductor nanowires, and graphene, among others. If the nanostructure is a carbon nanotube, the carbon nanotube can be classified by its chiral vector (n,m), which can indicate the orientation of the carbon hexagons. The orientation of carbon hexagons can affect interactions of the nanotube with other molecules, which in turn, can affect a property of the nanostructure.

In certain circumstances, the conductive material can be functionalized, for example, to include a coordinating group. The coordinating group can be a nitrogen heteroaryl group, for example, a pyridyl group. In one example, the conductive material can include pyridyl group functionalized carbon nanotubes. For example, the coordinating group can contain pyridyl groups, for example pyridyl groups functionalized carbon nanotubes. The pyridyl group contains a nitrogen group that has an sp² electronic configuration and one skilled in the art will understand that there are a number of other groups that can a display equivalent a pyridine group. As a result, the term pyridyl is indicative of a nitrogen based group capable or interacting with a metal similarly to a pyridine. There can also be multiple points of interaction between pyridyl groups. For example, a pyridyl can be a bipyridine or terpyridine group.

In certain circumstances, the palladium based catalyst can include Pd(II). For example, the palladium based catalyst can include a Pd(II) salt and a nitrite salt, a Pd(II) salt with a Cu(II) salt and a nitrite salt, or a Pd—Cu heterobimetallic complex and a nitrite salt.

In certain circumstances, the palladium based catalyst can include a nitrite salt, for example, an alkyl ammonium nitrite salt.

In certain circumstances, the sensor can include an ionic liquid solvent. The ionic liquid can be an electrolyte or a liquid salt. Non-limiting examples of ionic liquids include 1-allyl-3-methylimidazolium bromide, 1-allyl-3-methylimidazolium chloride, 1-benzyl-3-methylimidazolium hexafluorophosphate, 1-butyl-1-methylpyrrolidinium hexafluorophosphate. Other ionic liquids are also possible.

In certain circumstances, the sensor can include an oil or flexible polymer. The polymer can be a polysiloxane, polyoxyalkylene, polystyrene, polyamide, polyvinyl chloride, polyethylene, polyester, polypropylene, polycarbonate, polyacrylamide or polyvinyl alcohol. In certain circumstances, the sensor can include a polymer that has a glass transition temperature that is higher than the conditions in which the sensor is expected to operate.

In certain circumstances, the sensor can include a liquid material. For example, the liquid material can include an aromatic alcohol, for example, benzyl alcohol.

In certain circumstances, the electrodes contact the conductive materials. The gap between electrodes can range from 0.005 mm to 10 mm. The layer thickness of the conductive material and the palladium based catalyst can be between 0.01 μm to 5 μm. The mass ratio between the palladium based catalyst to the conductive material can be between 1:0.5 and 1:100. In some cases, the palladium based catalyst can be intrinsically conductive, and no additional conductive material need be added to create a chemiresistive sensor. For example, the electrode can include gold or chromium on a substrate. In one example, a gold layer can be deposited on a chromium layer.

In certain circumstances, the sensor can include a flow chamber for routing a gas from a sample to the conductive region.

In another aspect, a method of sensing an analyte can include exposing a sensor to a sample, the sensor including a conductive region in electrical communication with at least two electrodes, the conductive region including a conductive material and a palladium based catalyst material in contact with the conductive material, and measuring an electrical property at the electrodes.

In another aspect, a method of preparing a sensor can include forming a complex including a conductive region in electrical communication with at least two electrodes, the conductive region including a conductive material and a palladium based catalyst material in contact with the conductive material; and placing the conductive material in electrical communication with at least two electrodes.

In the methods described herein, the sensor can have one or more of the features described above.

In certain circumstances, the sample can include a gas. In certain circumstances, the analyte can include one or more of ethylene, 1-methylcyclopropene, butadiene, isoprene, carbon monoxide, acetylene, an alkene or an alkyne. The analyte can react with the catalyst to create a detectable change in resistance or conductivity in the sensor.

The sensor can detect the analyte low concentrations in a carrier gas, for example, in air. In certain circumstances, the analyte that can be detected can have a concentration of less than 100 ppm, less than 50 ppm, less than 1 ppm, less than 10 ppm or less than 1 ppm in the carrier gas. For example, the analyte can have a concentration of 500 ppb or less in the carrier gas.

In certain circumstances, the analyte can be an emission from a vegetable, fruit, flower, or plant.

Referring to FIG. 1C, sensor 10 can include a catalyst 30 including a conductive material between two electrodes 10, 20, in circuit 50. The catalyst 30 and electrodes 15, 20 can be on a substrate 40. The substrate can be an insulating material, for example, glass.

Referring to FIG. 23, a system for monitoring a gas can include a sample chamber 10, a gas carrier 12 and a detection chamber 14 that are fluidly connected. Detection chamber 14 can include a sensor 16, as described herein. A carrier gas can carry an analyte, if present, from the sample chamber 20 to the detection chamber 14.

Low-volatility solvents are required to avoid solvent evaporation during sensing experiments, precluding the direct translation of the reported solvent system by Grubbs and co-workers (^{t}BuOH/MeNO₂) to chemiresistive sensing. See, for example, Wickens, Z. K. et al. Aldehyde-Selective Wacker-Type Oxidation of Unbiased Alkenes Enabled by a Nitrite Co-Catalyst. Angew. Chemie—Int. Ed. 2013, 52, 11257-11260, which is incorporated by reference in its entirety. AgNO₂ with was substituted ⁿBu₄N[NO₂] to maintain higher solubility/uniformity. Low-volatility alcoholic solvents were screened by ¹H NMR using a model reaction with 1-heptene. The oxidation yield was calculated as the combined NMR yield of the oxidation products, heptanal and 2-heptanone (see Table 1). Solvent screening revealed that a range of low-volatility alcoholic solvents enabled alkene oxidation under ambient conditions (50-70% oxidation yield over 16 h), and that benzyl alcohol and 1-dodecanol afforded quantitative conversion of 1-heptene to its oxidized products. Removal of the alcohol moiety in the solvent had detrimental effects on total oxidation yield (<5% conversion when using toluene, benzyl cyanide, or acetophenone; Table 1, entries 7-9).

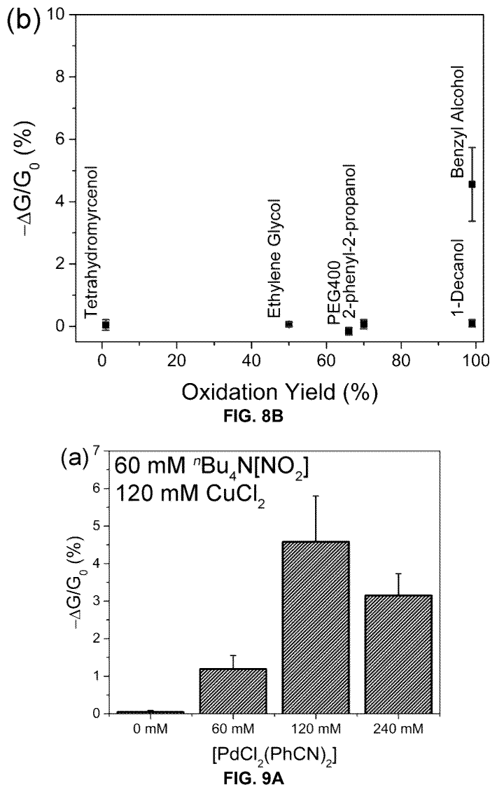

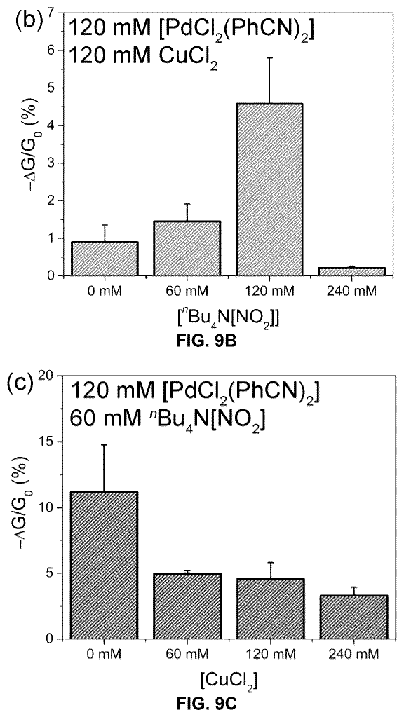

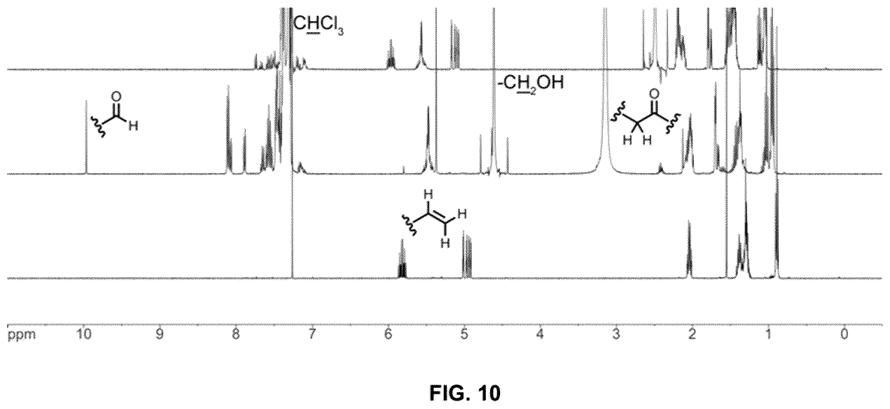

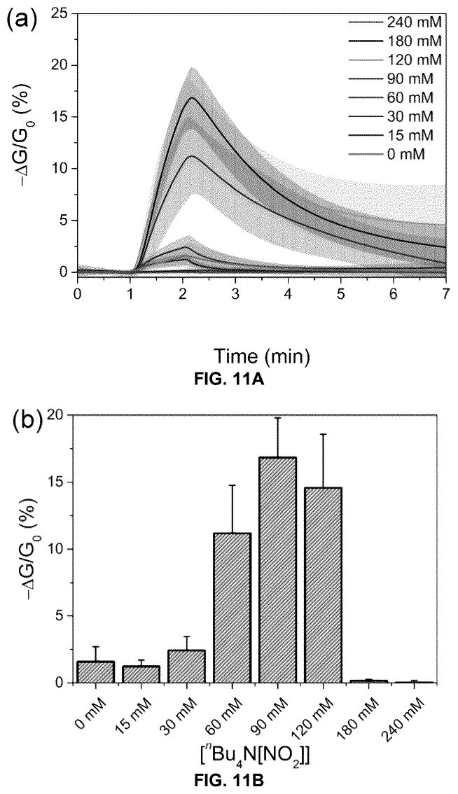

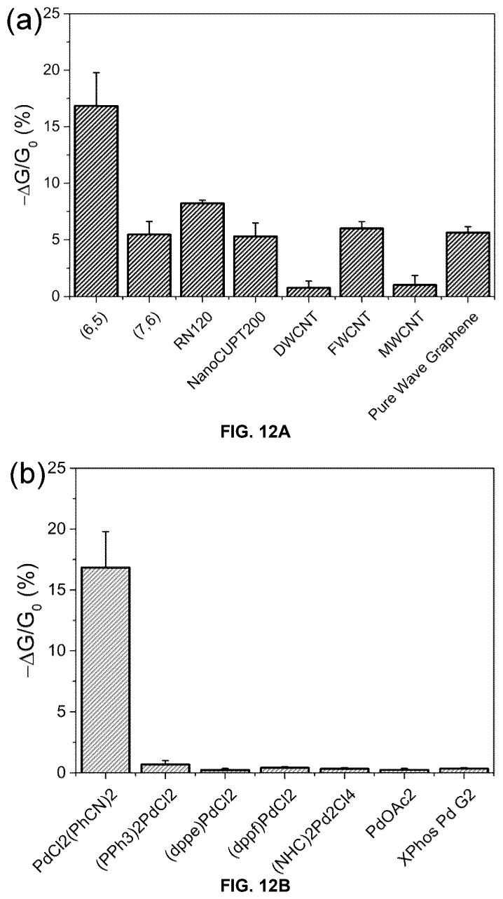

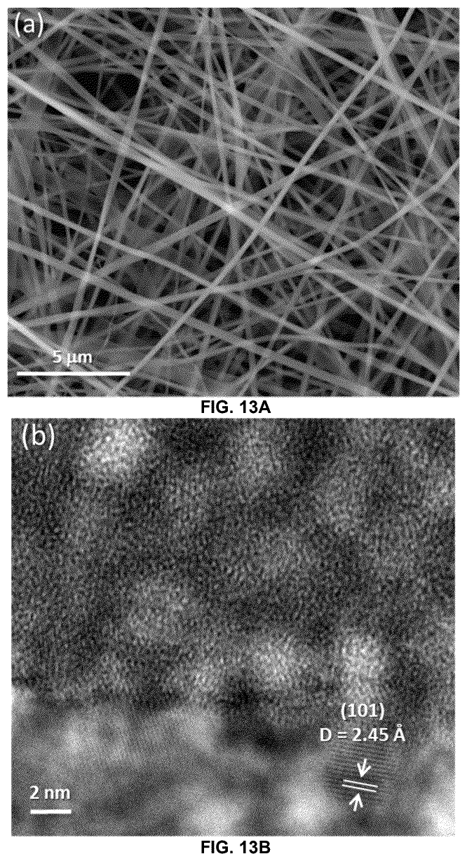

The oxidation yields were contrasted with the chemiresistive sensing response. As shown in FIG. 1C, a random nanowire matrix of (6,5) single-walled carbon nanotubes (SWCNTs) is deposited between two gold electrodes on an insulating glass substrate and then the catalytic reaction mixture is deposited on top of the SWCNTs. Sensor responses are measured as the change in conductance, ΔG/G₀, where ΔG is the change in conductance and G₀ is the baseline conductance in the absence of analyte. Devices were exposed to 1 min of 50 ppm ethylene in air and observed no correlation between ¹H NMR yields and sensing responses (FIGS. 8A-8B). The concentrations of each ingredient was varied using benzyl alcohol as the solvent, since it provided the strongest response from the solvent screen. Sensors having 120 mM concentrations of both [PdCl₂(PhCN)₂] and ⁿBu₄N[NO₂] afforded the largest responses to 50 ppm of ethylene (FIGS. 9A and 9B). Strikingly, removal of CuCl₂ co-catalyst resulted in substantial improvement (−11.2±3.6%, FIG. 9C). There is literature precedence for palladium-mediated olefin oxidation in the absence of a Cu source, and ¹H NMR studies confirm that aerobic oxidation of 1-heptene using benzyl alcohol as the solvent still occurs without CuCl₂ (FIG. 10). See, for example, Ning, X. S. et al. Tert-Butyl Nitrite: Organic Redox Cocatalyst for Aerobic Aldehyde-Selective Wacker-Tsuji Oxidation. Org. Lett. 2016, 18, 2700-2703; and Hu, K. F. et al. Tuning Regioselectivity of Wacker Oxidation in One Catalytic System: Small Change Makes Big Step. J. Org. Chem. 2018, 83, 11327-11332, each of which is incorporated by reference in its entirety. The concentration of ⁿBu₄N[NO₂] was optimized in the absence of CuCl₂. As shown in FIGS. 11A-11B, 90 mM ⁿBu₄N[NO₂] resulted in the strongest response of −16.8±3.0%. Various commercially available conductive nano-carbon sources and palladium sources were also screened, with no response improvement (FIGS. 12A-12B).

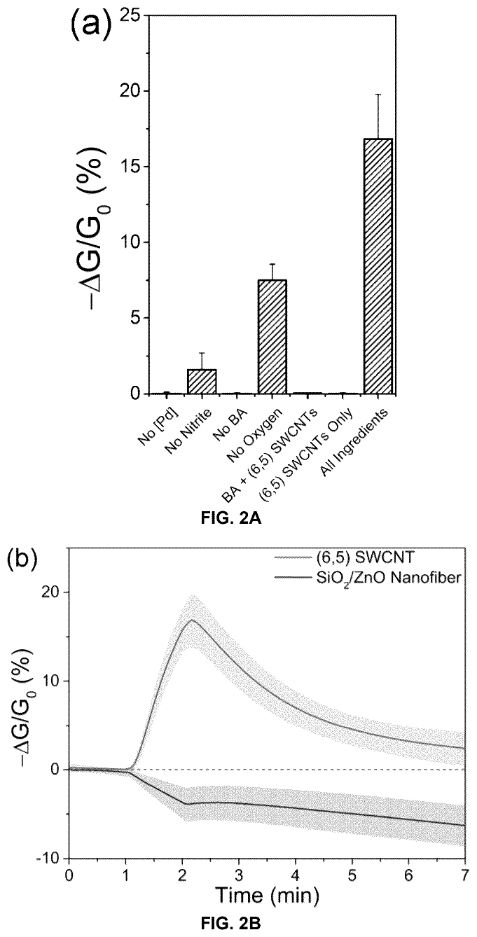

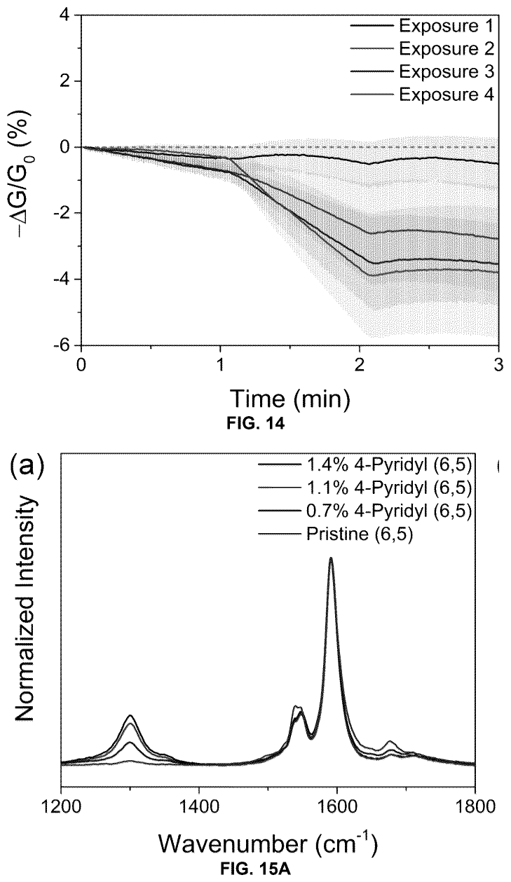

The proposed sensing mechanism was corroborated with some additional experiments. As shown in FIG. 2A, a detrimental effect on the sensing response is observed when vital components for Wacker oxidation are omitted. The use of (6,5) SWCNTs only and benzyl alcohol with (6,5) SWCNTs exhibited negligible response. For example, the generation of an n-dopant [i.e., Pd(0)] in situ can control the change in conductance observed. If so, then the direction of ΔG/G₀ should be reversed by replacing p-type SWCNTs with an n-type material. To test this hypothesis, n-type SiO₂/ZnO nanofibers were prepared (TEM images shown in FIGS. 13A-13B) according to modified literature procedures. See, for example, Shan, H. et al. Hierarchical Porous Structured SiO2/SnO2 Nanofibrous Membrane with Superb Flexibility for Molecular Filtration. ACS Appl. Mater. Interfaces 2017, 9, 18966-18976; and Song, G. et al. SiO2/ZnO Composite Hollow Sub-Micron Fibers: Fabrication from Facile Single Capillary Electrospinning and Their Photoluminescence Properties. Nanomaterials 2017, 7, which is incorporated by reference in its entirety. As shown in FIG. 2B, exposure to ethylene resulted in a clear increase in conductance for the n-type nanofibers, which is consistent with our proposed signal transduction mechanism. SiO₂/ZnO nanofiber-based sensors exhibited a dosimetric response and hysteresis upon repeated ethylene exposure (FIG. 14). Collectively these findings demonstrate that the in situ generation of Pd(0) via Wacker oxidation is responsible for a carrier reduction in the p-type SWCNTs, resulting in the observed change in conductance.

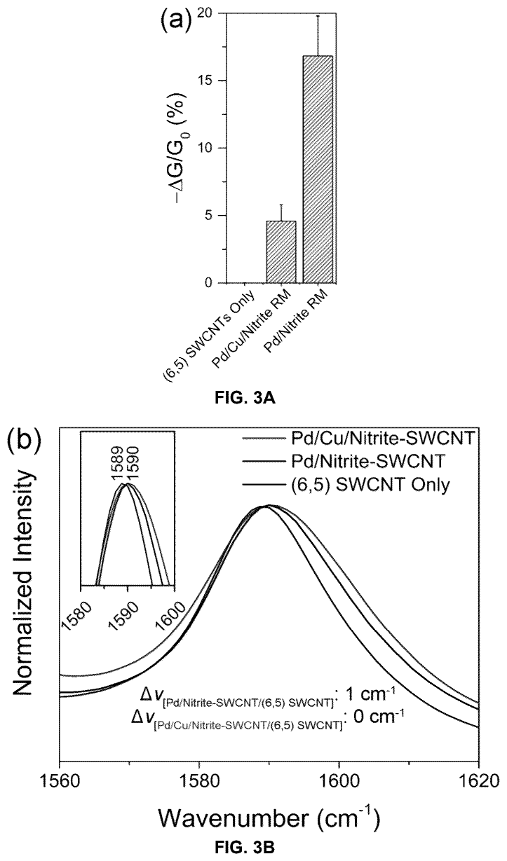

The role of CuCl₂ in the sensing response was probed based upon the postulate of Fu and co-workers that the Pd/Cu-catalyzed anti-Markovnikov Wacker oxidation is mediated by a heterobimetallic Pd—Cu complex. See, for example, Jiang, Y. Y. et al. Mechanism of Aldehyde-Selective Wacker-Type Oxidation of Unbiased Alkenes with a Nitrite Co-Catalyst. ACS Catal. 2015, S, 1414-1423, which is incorporated by reference in its entirety. Considering that the sensing mechanism occurs via Pd(0) n-doping of the SWCNT sidewalls, Pd(0) alone may be a better n-dopant than the Pd—Cu heterobimetallic complex. To test this hypothesis, devices were prepared containing pristine (6,5) SWCNTs wherein either 1 μL of the optimized Pd/Cu/nitrite or Pd/nitrite reaction mixture deposited on top. These devices were exhaustively subjected to 1-heptene in nitrogen (to prevent re-oxidation of the catalytic/dopant species) until solvent was fully evaporated. Using Raman spectroscopy, three different spots were interrogated per sample and the spectra were averaged. The G-band centred at ˜1590 cm⁻¹ corresponds to sp² C—C bond stretching and shifts toward lower frequencies in the presence of electron-donating (n-doping) molecules. See, for example, Rao, A. M. et al. Evidence for Charge Transfer in Doped Carbon Nanotube Bundles from Raman Scattering. Nature 1997, 388, 257-259, which is incorporated by reference in its entirety. FIG. 3A summarizes the sensing response using reaction conditions with and without CuCl₂ present. As shown in FIG. 3B, The G-band for the SWCNT sample subjected to the Pd/nitrite reaction mixture is shifted lower by 1 cm⁻¹, while SWCNTs exposed to the CuCl₂-containing reaction mixture are not noticeably shifted. This result is consistent with the notion that Pd(0) alone is a stronger n-dopant than the reduced Pd—Cu heterobimetallic complex, and may in part explain the observed difference in sensing response.

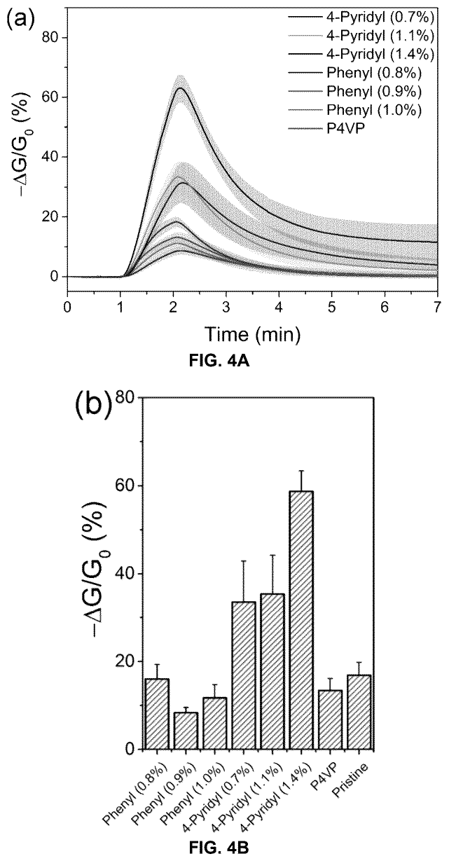

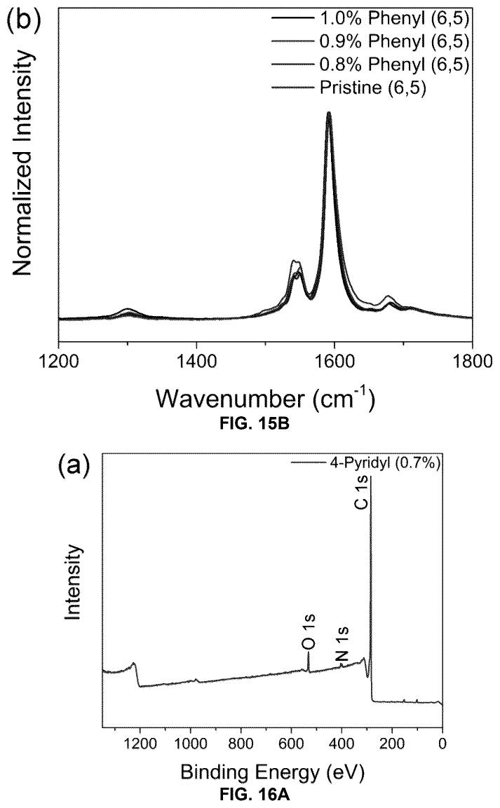

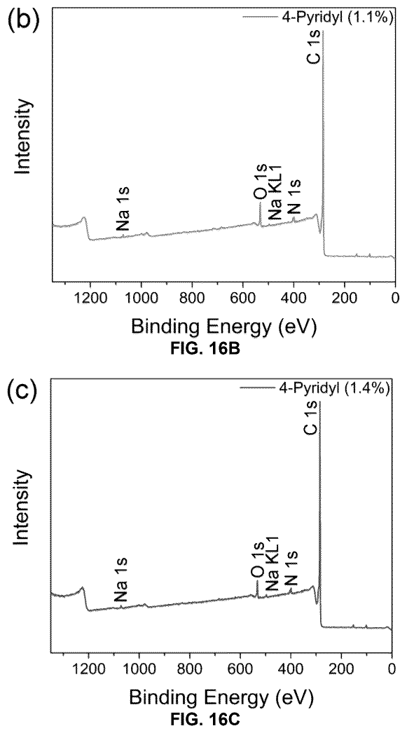

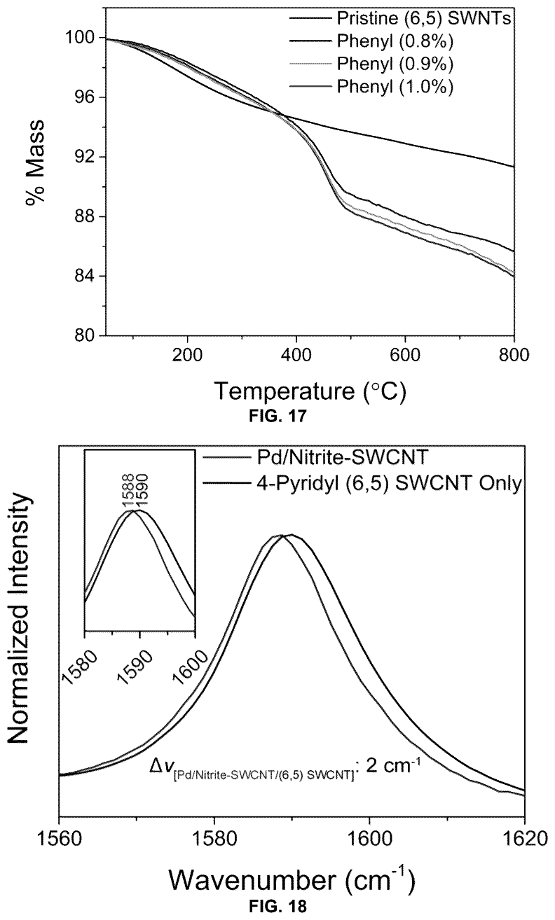

This mechanistic understanding can be leveraged to further improve the sensing response. appending coordinating groups to the SWCNT surface could ensure that the Pd(0) species are localized at the SWCNT sidewall for optimal carrier modulation. 4-Pyridyl-functionalized (6,5) SWCNTs was prepared using both covalent and non-covalent functionalization via iodonium salt reactions and poly(4-vinylpyridine) (P4VP) polymer wrapping, respectively. See, for example, He, M.; Swager, T. M. Covalent Functionalization of Carbon Nanomaterials with Iodonium Salts. Chem. Mater. 2016, 28, 8542-8549; and Yoon, B.; et al. Surface-Anchored Poly(4-Vinylpyridine)-Single-Walled Carbon Nanotube-Metal Composites for Gas Detection. Chem. Mater. 2016, 28, 5916-5924, each of which is incorporated by reference in its entirety. Covalently functionalized samples were characterized by Raman spectroscopy, X-ray photoelectron spectroscopy, and thermogravimetric analysis (FIGS. 15A-15C, 16A-16C and 17). As shown in FIGS. 4A-4B, 4-pyridyl covalent functionalization of (6,5) SWCNTs substantially improved the sensing response compared to pristine (6,5) nanotubes, with increasing degree of functionalization (up to 1.4 functional groups per 100 C atoms) proving beneficial. As a control experiment, (6,5) SWCNTs was covalently functionalized with phenyl groups and observed no improvement. See, for example, He, M.; Swager, T. M. Covalent Functionalization of Carbon Nanomaterials with Iodonium Salts. Chem. Mater. 2016, 28, 8542-8549, which is incorporated by reference in its entirety. Thus, the coordinating nitrogen atom is crucial for the observed effect. Non-covalently functionalized P4VP-coated SWCNTs did not improve device performance, which may be the result of the insulating nature of the polymer wrapping the SWCNT. As shown in FIG. 18, Raman G-band analysis reveals a shift of 2 cm⁻¹ upon Pd(0) doping. Although the magnitude of this shift is small, it is consistent with the notion that 4-pyridyl functionalized SWCNTs are more efficiently de-doped than pristine (6,5) SWCNTs.

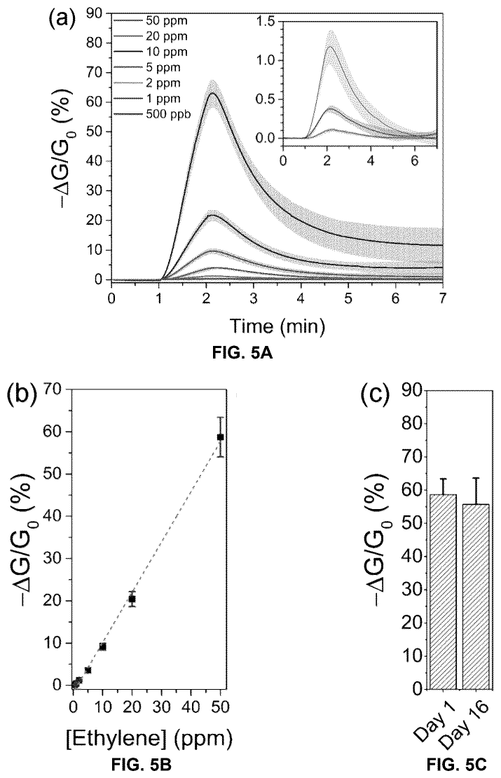

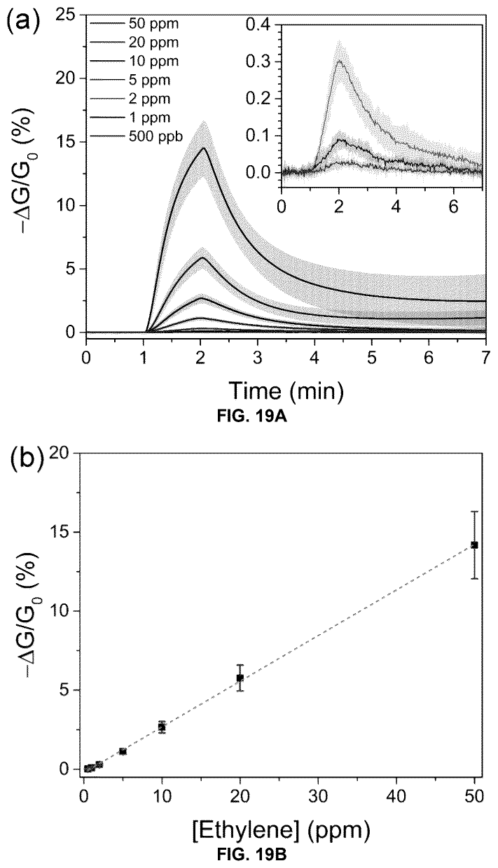

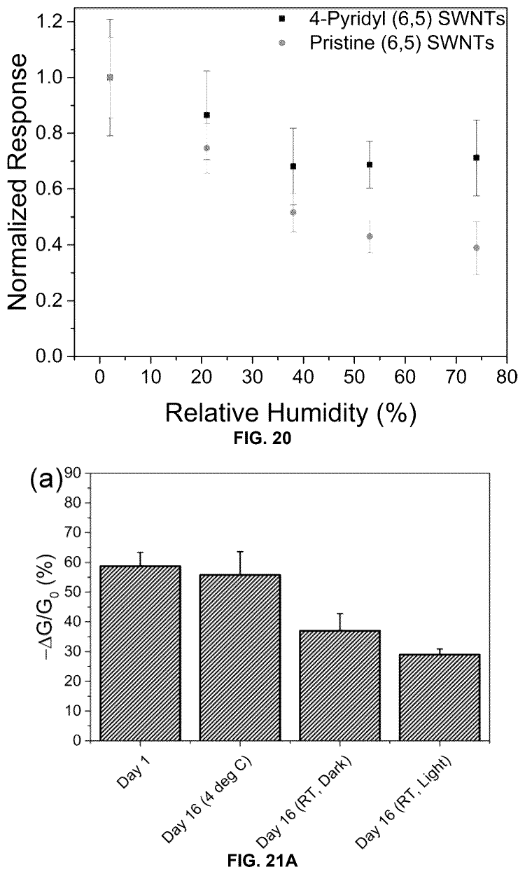

FIGS. 5A-5C depicts the sensitivity of our system using the optimized conditions with 4-pyridyl functionalized (6,5) SWCNTs. A clear signal is attained after one min exposure to 500 ppb ethylene in air and the responses are linear from 500 ppb to 50 ppm with a limit of detection (LOD) of 40 ppb. Using pristine (6,5) SWCNTs with other conditions the same, 500 ppb ethylene could also be detected (FIGS. 19A-19B). As shown in FIG. 20, the devices using 4-pyridyl functionalized SWCNTs exhibited the highest response to ethylene below 40% relative humidity (R.H.), and devices delivered a consistent response when operated at 40-80% R.H. In contrast, devices using pristine (6,5) SWCNTs exhibited a continued decrease in response as R.H. increased. Consistently the 4-pyridyl functionalized (6,5) SWCNTs based devices displayed superior stability with degradation in sensor performance after storage at 4° C. for 16 days in the dark (FIG. 5C). Some light sensitivity was observed (FIG. 21A), which is a known issue for nitrite-containing liquid samples. See, for example, Aminot, A.; Kérouel, R. Stability and Preservation of Primary Calibration Solutions of Nutrients. Mar. Chem. 1996, 52, 173-181, which is incorporated by reference in its entirety. For devices using pristine (6,5) SWCNTs, some performance degradation was observed even when stored at 4° C. (FIG. 21B). Overall, these results demonstrate that devices using 4-pyridyl functionalized (6,5) SWCNTs have superior sensitivity and stability compared to those using pristine (6,5) SWCNTs.

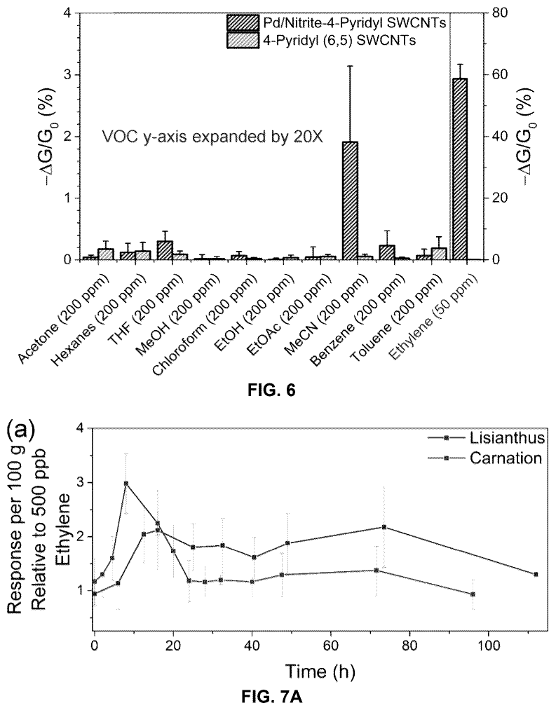

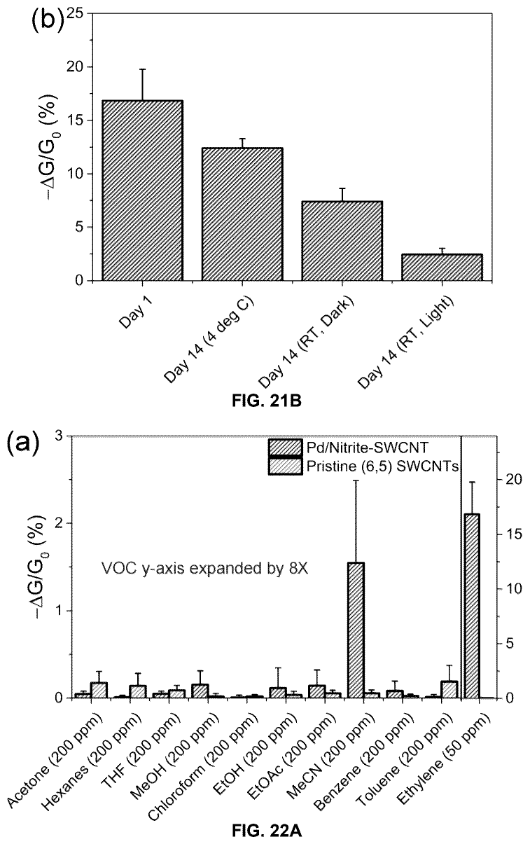

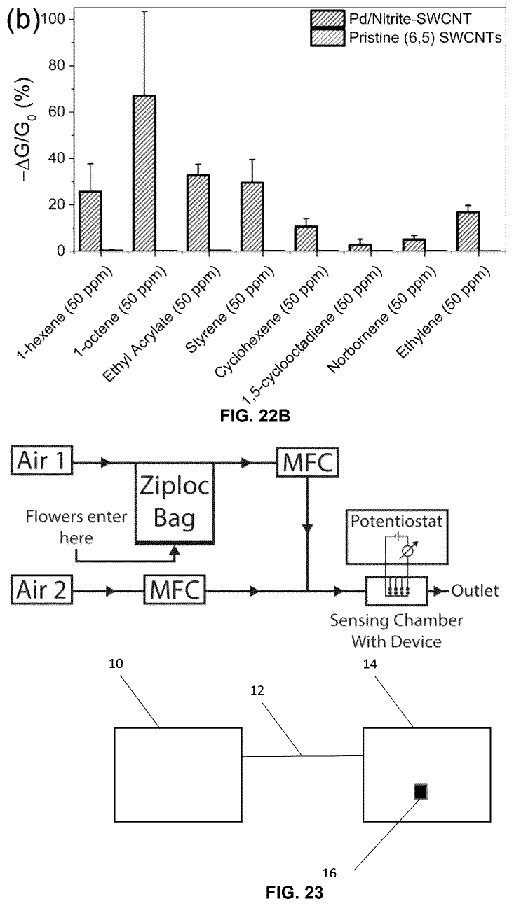

As shown in FIG. 6, the selectivity of our ethylene sensor using 4-pyridyl functionalized (6,5) SWCNTs is excellent when challenged against various volatile organic compounds (VOCs). Similar selectivity was observed using pristine (6,5) SWCNTs (FIG. 22A). The sensor responds to other terminal olefins and weakly to internal alkenes (FIG. 22B). Even though the chemical reactivity is not expected to be markedly different between terminal olefins, the response to 1-octene was almost twice that of 1-hexene. This suggests the partition coefficient between carrier gas and liquid influences the total available olefin for oxidation.

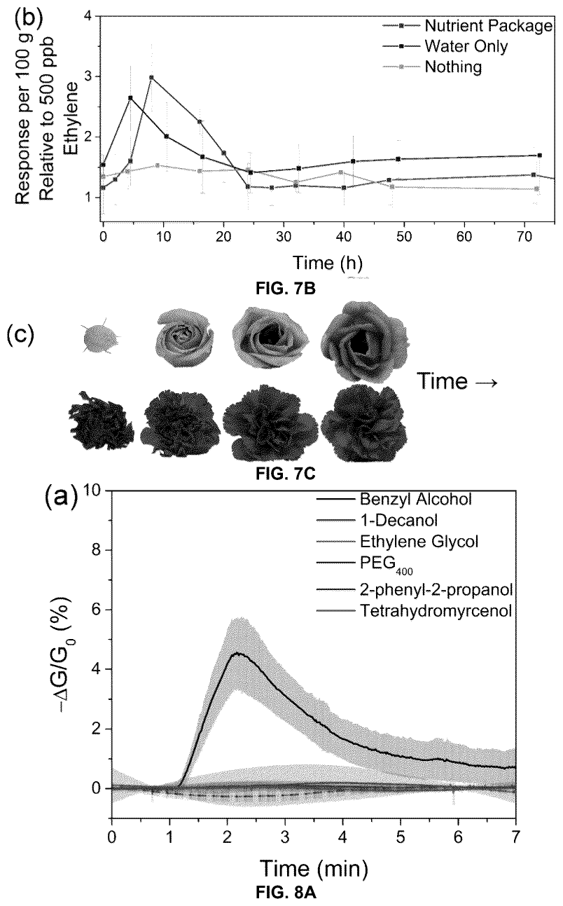

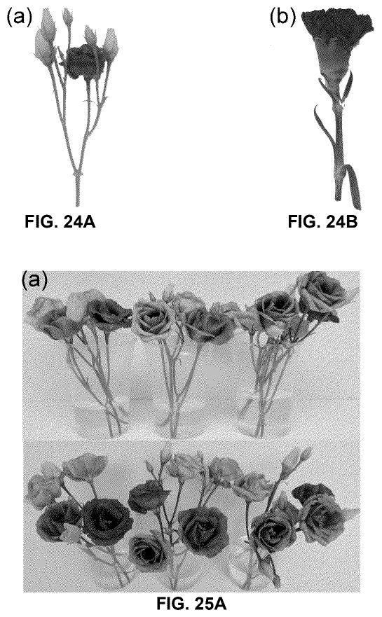

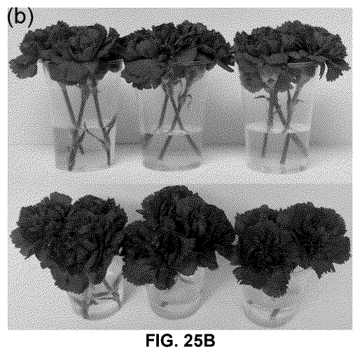

To demonstrate the utility of the sensory system for a challenging application the ability to monitor the ethylene evolution from flowers was demonstrated. The flowers were enclosed in a homemade chamber, allowing us to expose the sensor to flower volatiles in a controlled manner (FIG. 23). The device response to purple lisianthus flowers and red carnations at various time points is shown in FIG. 7A, and photographs of the flowers as received are shown in FIGS. 24A-24B. The intensities are given relative to the response to 500 ppb ethylene normalized to 100 g of flower. The flowers emitted ethylene concentrations up to ˜1.5 ppm, corresponding to an emission rate of about 320 nL·min⁻¹. Most carnation flowers bloomed within a day, while most lisianthus flowers bloomed over the period of a week. Compared to carnations, lisianthus flowers had greater variation in growth stage (FIGS. 25A-25B). These physical manifestations are contrasted with sensor measurements indicating rapid ethylene peaking over the span of several hours for carnations, and a gradual increase, plateau, and then decrease in ethylene evolution over the span of several days for lisianthus flowers. These ethylene profiles coincide with the observed blooming times for the flower populations, and are in agreement with the known low ethylene sensitivity of lisianthus flowers and high ethylene sensitivity of carnations. See, for example, Rybczyński, J. J. et al. The Gentianaceae—Volume 2: Biotechnology and Applications; 2015; and Woltering, E. J.; Van Doom, W. G. Role of Ethylene in Senescence of Petals—Morphological and Taxonomical Relationships. J. Exp. Bot. 1988, 39, 1605-1616, each of which is incorporated by reference in its entirety.

Intrigued by these results, a commercial flower nutrient package provided by the carnation supplier was tested to assess impact on the ethylene emission profile. The carnations were (i) left on the table without water, (ii) treated with water only, or (iii) treated with nutrient water. As shown in FIG. 7B, when the carnations are not treated with water, no change in ethylene emission is observed. Meanwhile, treatment with water results in peak ethylene emission about four hours earlier than treatment with nutrient water, which suggests that the nutrient packages do not strongly influence the ethylene emission profile. Gratifyingly, the sensor enables us to study dynamic plant processes mediated at low concentrations of the plant hormone, ethylene.

In summary, a carbon nanotube-based ethylene gas sensor that uses Wacker oxidation for the selective recognition of ethylene has been prepared. The devices are simple to prepare and allow us to detect ppb concentrations of ethylene in air. Sensitivity and stability can be enhanced using 4-pyridyl functionalized carbon nanotubes. This sensory system enables us to monitor flower senescence. When translating catalytic processes into chemiresistive sensing, several lessons are revealed: (1) Reaction chemoselectivity correlates well with sensor selectivity; (2) sensitivity is controlled by both chemical reactivity and dopant efficiency, which can be tuned by the judicious selection of the catalytic components, as well as the modification of semiconductor surface chemistry; and (3) the properties of the chemical transformation (such as air tolerance) and stability of the individual reaction components translate well to sensor attributes. A continued merger of catalytic oxidation processes and chemiresistive sensing as a potent system for the facile detection of additional analytes in air and under ambient conditions.

### Experimental

### General

(6,5) single-walled carbon nanotubes (SWCNTs) [lot #: MKBZ1159V; (6,5) chirality, ≥93% carbon as SWCNT; 0.7-0.9 nm diameter by fluorescence], PdCl₂(PhCN)₂, CuCl₂, and ⁿBu₄N[NO₂] were purchased from Sigma-Aldrich and used as received. All other palladium sources were purchased from Strem Co. and used as received. (7,6) SWCNTs [lot #: MKCJ6188; (7,6) chirality, ≥90% carbon basis; 0.83 nm average diameter], double-walled carbon nanotubes (lot #: MKBH3079V), few-walled carbon nanotubes (lot #: MKCD5241), and multi-walled carbon nanotubes (lot #: MKBB2306) were purchased from Sigma-Aldrich and used as received. Ultra-purified (UPT) SWCNTs (lot #: UPT-1210-133) were purchased from Nano-C and used as received. RN120 SWCNTs (batch #RNB738-120) and Pure Wave Graphene (batch #GRN32-075) were purchased from Nanointegris and used as received. For the ZnO nanofibers, poly(vinylpyrrolidone) (PVP, M_{w}=1.3 MDa), tetraethyl orthosilicate (TEOS), zinc nitrate hexahydrate (Zn(NO₃)₂·6H₂O) and poly(styrene sulfonate) (PSS, M_{w}=75,000 g·mol⁻¹) solution 30% w/v in H₂O were purchased from Sigma-Aldrich. Conc. HNO₃ and EtOH and were purchased from Synth, Brazil. These reagents were analytical grade and used without further purification. Purple lisianthus flowers were purchased freshly cut from Kendall Flower Shop (Boston, MA, USA). Red carnations were purchased from GlobalRose via Amazon.com, Inc. (ASIN: B00M8TP04M). Nutrient packages (FloraLife) were provided with the carnations and used as received. NMR was performed on a Bruker Avance III DRX 400 MHz instrument and shift-referenced to the residual solvent resonance. A gas cylinder containing 1% ethylene in nitrogen was purchased from Airgas (Airgas, Dorchester, MA) and equipped with a gas flow regulator to control the output pressure to ˜15 psi. Mass flow controllers (MFCs) were purchased from Alicat Scientific, with carrier gas flow rates (air or nitrogen) controlled using an MC-10SLPM-D/5M and ethylene flow rates controlled using an MC-10SCCM-D/5M. Flow rates were remotely controlled by connecting the MFCs to the sensing laptop via a 6′ USB-MD8-232 double-ended 8-pin mini-DIN to USB serial cable (Alicat Scientific) and using Flow Vision SC software (Alicat Scientific; available free of charge online) to change flow rates using a script. Resistance was measured using an Agilent Keysight 34970A potentiostat equipped with a 34901A 20-channel multiplexer (2/4-wire) module. The potentiostat was connected to the sensing laptop using an Agilent 82357B GPIB-USB Interface High-Speed USB 2.0 serial cable and controlled using BenchLink Data Logger 3 (available free of charge online). The scan rate was set to 1 scan/second. Raman spectra were collected using a Horiba Jobin-Yvon LabRam (Model HR 800) Raman confocal microscope with a 633 nm laser (1.4 μm spot size). Laser intensity was set to 10% for the 633 nm excitation wavelength. X-ray photoelectron spectroscopy (XPS) measurements were performed on a Thermo Scientific K-Alpha+ X-ray photoelectron spectrometer. Thermogravimetric analysis (TGA) was performed on a TA Discovery TGA system. Experiments were carried out under a nitrogen atmosphere with a gas flow rate of 25 mL/min. Samples were heated at a rate of 10° C./min to 800° C.

### ¹H NMR Solvent Screening Experiment

A glass vial equipped with a magnetic stir bar was charged with [PdCl₂(PhCN)₂] (6.8 mg, 17.6 μmol), CuCl₂·2H₂O (3 mg, 17.6 μmol), ⁿBu₄N[NO₂] (2.5 mg, 8.8 μmol), and solvent [benzyl alcohol, 1-decanol, ethylene glycol, 2-phenyl-2-propanol, polyethylene glycol (Mₙ=400; PEG₄₀₀), tetrahydromyrcenol, toluene, benzyl cyanide, or acetophenone](900 μL). 1-heptene (100 μL) was added and the reaction mixture was stirred for 16 h at RT under ambient conditions. The reaction mixture was filtered through a silica plug (˜1 cm in a glass pipette) and then an aliquot (100 μL) was diluted in CDCl₃ (600 μL) and subjected to ¹H NMR analysis (see Table 1 and FIGS. 8A-8B). Quantitation of the alkene and its oxidation products was determined by comparing the integrations for resonances at ˜5.80 ppm and ˜4.91-4.93 ppm (alkene protons), ˜9.75 ppm (aldehyde proton), and ˜2.39-2.43 ppm (—CH₂— methylene group adjacent to the carbonyl). For the ¹H NMR experiments involving no CuCl₂, an identical procedure was used to the one outlined above, except CuCl₂ was omitted. Workup and analysis were identical.

### Device Preparation

Glass slides (VWR microscope slides) were bath sonicated in acetone for 15 min and then dried with a stream of nitrogen. Using an aluminum mask, chromium (15 nm) followed by gold (50 nm) was deposited using a Thermal Evaporator (Angstrom Engineering), leaving a 1 mm gap between gold electrodes. Each glass slide had three devices, and each device had four channels. The devices were separated by scoring the glass with a diamond-tipped pen and then pulling the glass pieces apart. For pristine (6,5) SWCNTs: A stock solution of (6,5) SWCNTs (5 mg) was prepared in o-dichlorobenzene (DCB) (20 mL) by bath sonication at RT for 1 h, and then the stock solution was diluted 5-fold with fresh DCB and bath sonicated for another 30 min. 1 μL of the diluted (6,5) SWCNT dispersion was drop-casted in between the gold electrodes and dried at RT under house vacuum in a desiccator or vacuum oven. This drop-casting procedure was repeated until the resistance measured 1-3 kW using a handheld multimeter (Fluke 114 True RMS Multimeter).

### Ethylene Sensing Measurements

For a given reaction condition, 1 μL of the reaction mixture was added on top of the drop-casted SWCNTs and the device was enclosed in a homemade Teflon gas flow chamber. The carrier gas (air) was held constant at 1 L/min (except for sensitivity studies, where the flow rate was held constant at 2 L/min to obtain lower concentrations of ethylene), while the ethylene MFC was adjusted accordingly to control the concentration of ethylene (0.5-10 mL/min). The resistance of the device was measured over time (1 scan/sec), with typical parameters including 10 min equilibration time (for the baseline resistance to stabilize) followed by 1 min exposure to ethylene in air and then 5 min of recovery (air only). All presented data are given as the numeral average (N≥6) accompanied by the standard deviation.

### Gas Sensing Measurements (Interferents)

Device setup was identical to above, except that a gas generator (FlexStream, Kin-Tek) is used to produce gas vapours from liquid sources. A trace amount of analyte is emitted from a permeation tube diluted in air, which is further diluted with more air to adjust the concentration (in ppm) of analyte. Solvents and alkenes were calibrated by placing 2-3 mL of the liquid in the oven flow and measuring the mass loss after a known length of time at a constant temperature (usually 40° C.).

### Flower Studies

General. Flowers were stored in a ventilated office at 22° C. (˜23% relative humidity). To measure the response to flower volatiles, a homemade enclosure was prepared and is depicted in FIG. 23. A large Ziploc bag was cut in the bottom corners of the bag, such that the holes were smaller in diameter than 1 mL syringes. 1 mL syringes with the plunger and handle removed were placed in the incisions and then taped at the incision. Tubing was placed over the syringe barrel/tape and hose clamps were used to keep the apparatus together. Prior to exposure to flower volatiles, a blank injection with the empty, inflated Ziploc bag was tested to ensure no baseline change due to extraneous factors.

Measurement. Prior to each measurement, the flowers were weighed. If the flowers were treated with water, a paper towel was used to dry the immersed portion of the flower prior to mass measurement. Devices (N≥6) were prepared using 4-pyridyl functionalized (6,5) SWCNTs and 1 μL per channel of a benzyl alcohol solution containing 120 mM [PdCl₂(PhCN)₂] and 90 mM ⁿBu₄N[NO₂]. Devices were equilibrated under air at a flow rate of 200 mL/min and then exposed to flower volatiles for 1 min in air (200 mL/min). The device response was normalized to 100 g of flowers relative to 500 ppb of ethylene in air. The response to flower volatiles was measured every several hours.

Red Carnations. For each condition, 25-50 stems were cut until the flower was at a height of 10-12 cm. The mass of the flower populations ranged from ˜175-450 g. The carnations were treated with (i) nothing, (ii) water, or (iii) the nutrient packet dissolved in water. Plant nutrition packets (FloraLife) that came with the purchased flowers were dissolved in tap water (1 packet/L) and then distributed into plastic water cooler cups (˜75 mL per cup). For condition (ii), ˜75 mL of tap water-only was also used per cup. Each cup held at most three stems.

Purple Lisianthus. Stems were cut to a height of 10-15 cm. Each stem had 2-10 flower heads. A total of 52 stems with 256 flower heads (27 already opened or partially opened) were prepared with an initial mass of 293.2 g. Plant nutrition packets (FloraLife) were dissolved in tap water (1 packet/L) and then distributed into plastic water cooler cups (˜75 mL per cup). Each cup held at most four stems.

### Solvent Screening for Sensing Devices

A glass vial was charged with 120 mM [PdCl₂(PhCN)₂], 120 mM CuCl₂, and 60 mM ⁿBu₄N[NO₂] in solvent [benzyl alcohol, 1-decanol, ethylene glycol, 2-phenyl-2-propanol, PEG₄₀₀, tetrahydromyrcenol, or toluene] (300 μL). The reaction mixture was aliquoted onto the (6,5) SWCNTs (1 μL per device) and then resistance vs time was recorded.

Stoichiometry Optimization (with CuCl₂) for Devices

The initial conditions for the stoichiometry optimization experiment were 120 mM [PdCl₂(PhCN)₂], 120 mM CuCl₂, and 60 mM ⁿBu₄N[NO₂] in benzyl alcohol. To test the effect of reagent concentration on sensing response, two of the ingredients were held at the initial condition concentrations, while the concentration of the third ingredient was varied from 0-240 mM. To minimize weighing error, stock solutions of each reagent were prepared in benzyl alcohol and mixed in various ratios to produce the test condition (see Table S2). For the CuCl₂ stock solution, gentle heating with a heat gun to homogenize the solution was required prior to aliquoting it. After combining the ingredients, each reaction mixture was bath sonicated for 5 min prior to testing the sensing response.

### Conductive Nano-Carbon and Palladium Source Screening

Conductive Nano-Carbon Screening. The reaction mixture used in this experiment contained 120 mM [PdCl₂(PhCN)₂] and 90 mM ⁿBu₄N[NO₂] in benzyl alcohol. 1 μL of the carbon electrode material (dispersed in DCB) was drop-casted in between the gold electrodes and dried at RT under house vacuum. This drop-casting procedure was repeated until the resistance measured 1-3 kΩ using a handheld multimeter (Fluke 114 True RMS Multimeter). The reaction mixture was aliquoted onto the carbon electrode material (1 μL per device) and then resistance vs time was recorded.

Palladium Source Screening. The reaction mixture used in this experiment contained 120 mM palladium source and 90 mM ⁿBu₄N[NO₂] in benzyl alcohol. The mixture was sonicated for several min until homogenous and then used directly to prepare devices containing pristine (6,5) SWCNTs. Resistance vs time was recorded.

### Raman G-Band Experiment

Pristine or 4-pyridyl functionalized (6,5) SWCNTs were drop-casted and dried in between gold electrodes until black residue (i.e., the carbon nanotubes) was visible by eye. These SWCNT thin films were treated with (i) nothing, (ii) 1 μL of [PdCl₂(PhCN)₂]/CuCl₂/Bu₄N[NO₂] (120 mM/120 mM/60 mM), or (iii) 1 μL of [PdCl₂(PhCN)₂]/ⁿBu₄N[NO₂] (120 mM/90 mM). Samples (including the SWCNT-only samples) were exposed to 1-heptene in nitrogen at a flow rate of 3 L/min (using the gas generator) until solvent was evaporated. Raman analysis was performed using a 532 nm laser (10% intensity, 5 accumulations, and 3 sec integration time). For each sample, three different spots were examined and then the spectra were averaged.

### SiO₂/ZnO Nanofiber Fabrication

Zn(NO₃)₂·6H₂O (4.8% w/w) and PVP (9.5% w/w) were dissolved in DMF and stirred for 6 h at RT. Separately, TEOS (12.5% w/w) was dissolved in HNO₃/EtOH (100:1 TEOS:HNO₃ molar ratio) and stirred for 2 h at RT. The TEOS solution was added to the Zn/PVP solution (Si²⁺/Zn²⁺ molar ratio of 3.5:1) and stirred for 6 h RT. Electrospinning was performed on this solution by placing in a glass syringe and then applying voltage (12 kV) with a flow rate of 0.3 mL·h⁻¹ at a working distance of 6 cm. Nanofiber fabrication was followed by annealing at 60° C. for 6 h and calcination at 500° C. for 4 h. Field-emission gun scanning electron microscopy (FEG-SEM) and high-resolution transmission electron microscopy (HR-TEM) were performed with a JEOL-JSM 6701F and FEI Tecnai G2F20 microscope, respectively. The SiO₂/ZnO nanofibers were suspended in PSS aqueous solution (50 μg·mL⁻¹ of PSS) for a final concentration of 10 mg·mL⁻¹ of nanofiber and then 1 μL of the suspension was drop-casted onto the gold electrodes for gas sensing.

### Synthesis of Covalently Functionalized (6,5) SWCNTs

4-pyridyl functionalized SWCNTs were prepared according to literature procedures, and the same procedure was adapted for the preparation of phenyl functionalized SWCNTs. See, for example, He, M.; Swager, T. M. Covalent Functionalization of Carbon Nanomaterials with Iodonium Salts. Chem. Mater. 2016, 28, 8542-8549, which is incorporated by reference in its entirety. In a glovebox, a 40 mL glass vial was charged with a Teflon-coated stir bar, (6,5)-SWCNTs (5 mg, 0.42 mmol C), and THF (20 mL). Appropriate amount of freshly prepared solution of sodium naphthalide (0.021, 0.042, or 0.083 mmol) was transferred to the SWCNT suspension. The vial was capped, sealed tightly with electrical tape, removed from the glovebox, and sonicated in an ultrasonic bath for 30 min. The greenish colour disappeared upon sonication, indicating transfer of electrons from the naphthalide to the SWCNTs. After sonication, the vial was transferred to the glovebox and a solution of phenyl(2,4,6-triisopropylphenyl)iodonium triflate (0.021, 0.042 or 0.083 mmol) in THF (2 mL) was added. The reaction was stirred at RT for 3 h. The reaction was removed from the glovebox and filtered through a 0.2 μm nylon membrane. The collected SWCNT product was re-dispersed in 10:1 EtOH:H₂O (100 mL), sonicated for 30 min, and filtered through a 0.2 μm nylon membrane. This washing step was performed a total of four times. The resulting phenyl-functionalized (6,5) SWCNT product was dried in a vacuum oven at 60° C. for 24 h.

### Covalently Functionalized (6,5) SWCNT Screening

The reaction mixture used in this experiment contained 120 mM [PdCl₂(PhCN)₂] and 90 mM ⁿBu₄N[NO₂] in benzyl alcohol. 1 μL of the covalently functionalized (6,5) SWCNTs in EtOH was drop-casted in between the gold electrodes and dried at RT under house vacuum. This drop-casting procedure was repeated until the resistance measured 0.1-0.3 MΩ using a handheld multimeter (Fluke 114 True RMS Multimeter). The reaction mixture was aliquoted onto the SWCNTs (1 μL per device) and then resistance vs time was recorded.

TABLE 1
Reaction screening via ¹H NMR in CDCl₃ of various solvents for
the conversion of 1-heptene to heptanal or 2-heptanone using 3.9 mol %
[PdCl₂(PhCN)₂], 3.9 mol % CuCl₂•2H₂O, and 2 mol % ⁿBu₄N(NO₂)
under ambient conditions.

| Solvent | Heptanal | 2-Hexanone | Total Oxidation Yield* |
|---|---|---|---|
| Benzyl Alcohol | 28% | 72% | Quant. |
| 1-decanol | 2% | 98% | Quant. |
| Ethylene glycol | <1% | 50% | 50% |
| 2-phenyl-2-propanol | 2% | 68% | 70% |
| PEG₄₀₀ | 6% | 60% | 66% |
| Tetrahydromyrcenol | 1% | 0% | 1% |
| Toluene | 3% | 0% | 3% |
| Benzyl Cyanide | <1% | 0% | <1% |
| Acetophenone | <1% | 0% | <1% |

*Sum of heptanal and 2-heptanone yields.

TABLE 2
Stock solutions of [PdCl₂(PhCN)₂] (480 mM), CuCl₂ (480
mM), and ⁿBu₄N[NO₂] (240 mM) in benzyl alcohol were
mixed in various ratios to produce the test conditions in FIGS. 9A-9C.

| Condition | [PdCl₂(PhCN)₂] | | CuCl₂ | | ⁿBu₄N[NO₂] | | Benzyl Alcohol | |
|---|---|---|---|---|---|---|---|---|
| Initial Conditions | 20 | μL | 20 | μL | 20 | μL | 20 | μL |
| Pd Optimization (240 mM) | 40 | μL | 20 | μL | 20 | μL | 0 | μL |
| Pd Optimization (60 mM) | 10 | μL | 20 | μL | 20 | μL | 30 | μL |
| Pd Optimization (0 mM) | 0 | μL | 20 | μL | 20 | μL | 40 | μL |
| Cu Optimization (240 mM) | 20 | μL | 40 | μL | 20 | μL | 0 | μL |
| Cu Optimization (60 mM) | 20 | μL | 10 | μL | 20 | μL | 30 | μL |
| Cu Optimization (0 mM) | 20 | μL | 0 | μL | 20 | μL | 40 | μL |
| Nitrite Optimization (240 mM) | 20 | μL | 20 | μL | 40 | μL | 0 | μL |
| Nitrite Optimization (60 mM) | 20 | μL | 20 | μL | 10 | μL | 30 | μL |
| Nitrite Optimization (0 mM) | 20 | μL | 20 | μL | 0 | μL | 40 | μL |

Details of one or more embodiments are set forth in the accompanying drawings and description. Other features, objects, and advantages will be apparent from the description, drawings, and claims. Although a number of embodiments of the invention have been described, it will be understood that various modifications may be made without departing from the spirit and scope of the invention. It should also be understood that the appended drawings are not necessarily to scale, presenting a somewhat simplified representation of various features and basic principles of the invention.

1-30. (canceled)

31. A sensor comprising:
a conductive region in electrical communication with at least two electrodes, the conductive region including a conductive material and a palladium-based catalyst material in contact with the conductive material, wherein the palladium-based catalyst includes a Pd(II) salt and a nitrite salt, a Pd(II) salt with a Cu(II) salt and a nitrite salt, and/or a Pd—Cu heterobimetallic complex and a nitrite salt.

32. The sensor of claim 31, wherein the conductive material includes a p-type conductor or an n-type conductor.

33. The sensor of claim 31, wherein the conductive material includes a carbon nanotube, a carbon nanotube including a coordinating group, graphite, or graphene.

34. The sensor of claim 33, wherein the carbon nanotube including the coordinating group includes a pyridyl functionalized carbon nanotube.

35. The sensor of claim 31, wherein the conductive material includes silica/zinc oxide nanoparticles and/or nanofibers.

36. The sensor of claim 31, wherein the palladium-based catalyst includes Pd(II).

37. The sensor of claim 31, wherein the palladium-based catalyst includes an alkyl ammonium nitrite salt.

38. The sensor of claim 31, wherein the conductive material includes a metal oxide.

39. The sensor of claim 31, wherein the conductive material includes an inorganic semiconductor.

40. The sensor of claim 31, wherein the sensor includes an ionic liquid solvent.

41. The sensor of claim 31, wherein the sensor includes an oil or a flexible polymer.

42. The sensor of claim 31, wherein the sensor includes a liquid material.

43. The sensor of claim 42, wherein the liquid material includes an aromatic alcohol.

44. The sensor of claim 31, wherein the sensor includes a polymer that has a glass transition temperature that is higher than the conditions in which the sensor is expected to operate.

45. The sensor of claim 31, wherein at least one electrode includes gold or chromium.

46. The sensor of claim 31, further comprising a flow chamber for routing a gas including a sample to the conductive region.

47. A method of sensing an analyte, comprising:
exposing a sensor to a sample, the sensor including:
a conductive region in electrical communication with at least two electrodes, the conductive region including a conductive material and a palladium-based catalyst material in contact with the conductive material, wherein the palladium-based catalyst includes a Pd(II) salt and a nitrite salt, a Pd(II) salt with a Cu(II) salt and a nitrite salt, and/or a Pd—Cu heterobimetallic complex and a nitrite salt; and
measuring an electrical property at the electrodes.

48. The method of claim 47, wherein the sample is a gas.

49. The method of claim 47, wherein the electrical property is resistance or conductance.

50. The method of claim 47, wherein the analyte is ethylene.

51. The method of claim 47, wherein the analyte is 1-methylcyclopropene.

52. The method of claim 47, wherein the analyte is butadiene.

53. The method of claim 47, wherein the analyte is isoprene.

54. The method of claim 47, wherein the analyte is carbon monoxide.

55. The method of claim 47, wherein the analyte is acetylene.

56. The method of claim 47, wherein the analyte contains an alkene.

57. The method of claim 47, wherein the analyte contains an alkyne.

58. The method of claim 47, wherein the analyte is an emission from a vegetable, a fruit, a flower, or a plant.

59. A method of preparing a sensor comprising:
forming a complex including a conductive region in electrical communication with at least two electrodes, the conductive region including a conductive material and a palladium-based catalyst material in contact with the conductive material, wherein the palladium-based catalyst includes a Pd(II) salt and a nitrite salt, a Pd(II) salt with a Cu(II) salt and a nitrite salt, and/or a Pd—Cu heterobimetallic complex and a nitrite salt; and
placing the conductive material in electrical communication with at least two electrodes.
